(19) United States
(12) Patent Application Publication
(10) Pub. No.: US 2025/0313610 A1
(43) Pub. Date: Oct. 9, 2025

(54) USE OF APOLIPOPROTEIN H IN MANUFACTURE OF MEDICAMENT FOR PREVENTION AND/OR TREATMENT OF FATTY LIVER DISEASE AND RELATED DISEASES

(71) Applicant: Yaming LIU, Changchun (CN)

(72) Inventor: Yaming LIU, Changchun (CN)

(21) Appl. No.: 19/241,432

(22) Filed: Jun. 18, 2025

Related U.S. Application Data
(63) Continuation of application No. PCT/CN2023/090101, filed on Apr. 23, 2023.

(30) Foreign Application Priority Data
Apr. 19, 2023 (CN) 202310420781.9

(51) Int. Cl. C07K 14/775; A61K 38/00

(52) U.S. Cl. CPC C07K 14/775; A61K 38/00

(57) ABSTRACT

Use of apolipoprotein H (APOH) in the manufacture of medicaments for prevention and/or treatment of fatty liver disease and related diseases is provided. Using wild type C57BL/6 mice as controls, the serum transaminase level in peripheral blood of 10-week-old ApoH gene knockout mice (C57BL/6 ApoH^{-/-}) was assessed. The results showed that the levels of alanine aminotransferase (ALT) and aspartate aminotransferase (AST) are significantly increased. The triglyceride (TG) level was further assessed in the liver and the results showed that the TG level was significantly increased in the liver of ApoH^{-/-} mice. Based on the findings that low APOH expression induces steatohepatitis, promotes the progression of chronic liver diseases (CLDs), and is related to the prognosis of end-stage liver diseases, it is conducive to the development of precise targeted therapeutic drugs for treating fatty liver disease and improving the prognosis of the end-stage liver diseases.

### CROSS-REFERENCE TO RELATED APPLICATIONS

This application is a Continuation of International Application No. PCT/CN2023/090101, filed on Apr. 23, 2023, which claims priority to Chinese Patent Application No. 202310420781.9, filed on Apr. 19, 2023, the entire contents of each of which are hereby incorporated by reference.

### SEQUENCE LISTING

The instant application contains a Sequence Listing which is submitted electronically in XML format and is hereby incorporated by reference in its entirety. The XML copy, created on Jun. 12, 2025, is named “2025 Jun. 12-Sequence list-63501-H001US00”, and is 8,396 bytes in size.

### TECHNICAL FIELD

The present disclosure relates to the technical field of biomedicine technology, and in particular, to use of apolipoprotein H in the manufacture of a medicament for the prevention and/or treatment of metabolic dysfunction-associated steatotic liver disease (MASLD) and related diseases.

### BACKGROUND

Currently, the prevalence of MASLD among adults in China is close to 30%, making it the most common chronic liver diseases (CLDs) surpassing viral hepatitis in incidence. During the progression of CLDs, the occurrence of fatty liver accelerates the progress towards end-stage liver diseases, including cirrhosis and hepatocellular carcinoma (HCC). However, the pathogenic mechanism of this spectrum remains unclear.

Apolipoprotein H (APOH), also known as beta2-glycoprotein | (β2-GPI), is primarily synthesized and secreted by hepatocytes. The function of APOH is involved in lipid metabolism. Some studies reported that APOH plays a key role as an acute phase protein during viral infections. Our previous studies clarified that APOH was mainly involved in the regulation of lipid metabolism during the progression of CLDs. However, the exact mechanism of APOH in MASLD is still unclear.

### SUMMARY

One or more embodiments of the present disclosure provide the use of apolipoprotein H (APOH) in the manufacture of a medicament for the prevention and/or treatment of MASLD and related diseases. One or more embodiments of the present disclosure disclose the mechanism of fatty liver in the pathophysiological process of CLDs. One or more embodiments of the present disclosure also provide a novel method for preventing and/or treating MASLD and related diseases, such method comprising administering the medicament disclosed herein to patients. In some embodiments, the medicament comprises APOH or APOH gene, and the medicament is used to increase an expression level of apolipoprotein H in the liver. In some embodiments, the medicament further comprises a medically acceptable excipient, and the medicament is in the form of an injectable, a tablet, a granule, an oral formulation, or a genetically engineered drug.

### BRIEF DESCRIPTION OF THE DRAWINGS

The present disclosure will be further illustrated by way of exemplary embodiments, which will be described in detail through the accompanying drawings, wherein:

FIG. 1 is a schematic diagram of a guide RNA (gRNA) of ApoH gene in mouse according to some embodiments of the present disclosure;

FIG. 2 is a schematic diagram of the gene sequence from the liver of ApoH^{-/-} mouse model according to some embodiments of the present disclosure;

FIGS. 3A-3D are charts illustrating the phenotype identification results of mouse model according to some embodiments of the present disclosure;

FIGS. 4A-4K show the levels of APOH gene in the liver tissues of patients with CLDs and HCC, and survival analysis of HCC patients according to some embodiments of the present disclosure; and

FIGS. 5A-5C are charts illustrating the results of phenotypic identification from a fatty liver mouse model according to some embodiments of the present disclosure.

### DETAILED DESCRIPTION

In order to provide a clearer understanding of the technical solutions of the embodiments described in the present disclosure, a brief introduction to the drawings required in the description of the embodiments is given below. It is evident that the drawings described below are merely some examples or embodiments of the present disclosure, and for those skilled in the art, the present disclosure may be applied to other similar situations without exercising creative labor, unless otherwise indicated or stated in the context, the same reference numerals in the drawings represent the same structures or operations.

Set forth in the present disclosure and the claims, unless explicitly indicated otherwise in the context, words such as “one”, “a”, “an”, and/or “the” do not specifically denote the singular form and may also include the plural form. In general, the terms “comprising” and “including” only suggest the inclusion of steps and elements that have been explicitly identified, and these steps and elements do not constitute an exclusive listing; methods may also include other steps or elements.

Unless otherwise defined, all technical and scientific terms used in the present disclosure have the same meaning as typically understood by those of ordinary skill in the art to which the present disclosure pertains.

One or more embodiments of the present disclosure provide use of apolipoprotein H in the manufacture of a medicament for prevention and/or treatment of MASLD and related diseases.

Apolipoprotein H (APOH), also known as beta2-glycoprotein | (β2-GPI), is synthesized and secreted by hepatocytes. The function of APOH is involved in lipid metabolism. Some studies reported that APOH plays a key role as an acute phase protein during viral infections. APOH provides a new therapeutic target for the development of medicaments against MASLD and related diseases. Specifically, the expression level of APOH can be elevated by implementing targeted therapy on liver to regulate hepatic lipid metabolism and restore homeostasis, thereby aiding in the treatment of MASLD and related diseases.

In some embodiments, the medicament is a therapeutic or prophylactic drug. In some embodiments, the medicament comprises APOH protein or APOH gene that can increase APOH levels at the protein and gene levels in the liver. For example, it works by targeting the liver through exogenous administration of APOH complexes or genetically engineered drugs. Alternatively, the exogenous use of hepatoprotective agents or the use of artificial liver support systems are used to restore or improve hepatocyte function, thereby facilitating an increase in APOH protein synthesis. In addition, intervention strategies aimed at modulating the gut-liver axis may be used to alleviate liver inflammation and damage and to restore or improve hepatocyte function, thereby facilitating an increase in the synthesis of APOH protein.

In some embodiments, the medicament further comprises a medically acceptable excipient, and the medicament is in the form of an injectable, a tablet, a granule, an oral formulation, or a genetically engineered drug. In some embodiments, MASLD and related diseases include, but are not limited to, metabolic and alcoholic associated liver diseases (MetALD), viral hepatitis, autoimmune liver disease, liver fibrosis, liver cirrhosis, HCC, etc.

One or more embodiments of the present disclosure provide a medicament for prevention and/or treatment of MASLD and related diseases. In some embodiments, the medicament comprises APOH, or the medicament comprises a reagent that promotes expression of APOH protein in vivo. The medicament is used to increase APOH levels in the liver. In some embodiments, the medicament further comprises a medically acceptable excipient, and the medicament is in the form of an injectable, a tablet, a granule, an oral formulation, or a genetically engineered drug.

One or more embodiments of the present disclosure provide the use of APOH in the manufacture of a medicament for preventing and/or treating MASLD and related diseases. In some embodiments, APOH is used to regulate lipid metabolism in the liver, reduce the degree of hepatic steatosis, and alleviate inflammatory injury and reverse fibrosis, to improve the prognosis of patients with end-stage liver disease and prolong the survival time of the patients.

In some embodiments, the medicament comprises APOH protein or APOH gene, which can regulate the APOH levels in the liver. Specifically, the medicament uses APOH as a therapeutic target to regulate APOH production in the liver according to the different status of individual patient. This regulation helps maintain APOH level within the normal range, thereby contributing to the restoration of homeostasis in hepatic lipid metabolism.

Using wild type (WT) C57BL/6 mice as controls, the serum transaminase levels in the peripheral blood of 10-week-old ApoH gene knockout mice (C57BL/6 ApoH^{-/-}) were assessed. The results showed that the levels of alanine aminotransferase (ALT) and aspartate aminotransferase (AST) were significantly increased (in acute and chronic liver diseases, a ratio of AST/ALT less than 1 usually indicates mild liver injury; and a ratio of AST/ALT greater than 1 indicates severe liver injury). Further analysis showed that triglyceride (TG) levels were significantly elevated in the liver of ApoH^{-/-} mice. ApoH^{-/-} mice exhibit spontaneous steatohepatitis. Above all, we conclude that exogenous administration may be employed to increase the level of APOH in the liver and promote the restoration of homeostasis in hepatic lipid metabolism and alleviate hepatic steatosis, which contributes to the recovery of liver function.

One or more embodiments of the present disclosure provide use of APOH protein in the manufacture of biological detection reagents for different stages of MASLD and related diseases. One or more embodiments of the present disclosure also provide the use of APOH levels in the serum as a diagnostic marker for evaluating the progression of hepatic steatosis. In some embodiments, APOH is used as a biomarker for MASLD and related diseases. Specifically, the detection reagents may be used to assess the severity of fatty liver according to APOH expression at protein and gene levels.

By analyzing the liver transcriptomic datasets from CLDs patients, the results showed that the expression of APOH gene in the liver of patients with non-alcoholic steatohepatitis (NASH) and chronic hepatitis B were significantly decreased with the progression of liver fibrosis. The difference was statistically significant.

Further analysis of the liver transcriptomic datasets from HCC patients revealed that the expression of APOH gene was significantly decreased in the liver tumor tissues compared to the adjacent normal liver tissues. Furthermore, further survival analysis indicated that the downregulation of APOH gene is negatively correlated with prognosis, and the differential survival was observed between the low and high APOH expression groups.

Some studies reported that when synthesis and secretion of APOH are reduced, its expression level can be increased by supplementation with exogenous drugs or by improving liver function, thereby treating MASLD and related diseases.

One or more embodiments of the present disclosure also provide a method for preparing, using APOH, a medicament for prevention and/or treatment of MASLD and related diseases.

In some embodiments, APOH protein is used as a biological detection reagent for detecting different stages of MASLD and related diseases. Specifically, the detection reagent is used to assess the severity of fatty liver based on the APOH expression at protein and gene levels.

In some embodiments, APOH serves as a biomarker for MASLD and related diseases.

In some embodiments, the medicament is a therapeutic drug or a prophylactic drug.

In some embodiments, the medicament comprises APOH or APOH gene, which may be used to increase APOH production in the liver.

In some embodiments, the medicament also comprises a medically acceptable excipient, and the medicament is in the form of an injectable, a tablet, a granule, an oral formulation, or a genetically engineered drug.

In some embodiments, MASLD and related diseases include, but are not limited to, MetALD, viral hepatitis, autoimmune liver disease, liver fibrosis, liver cirrhosis, and HCC, et al.

The embodiments of the present disclosure have at least the following beneficial effects: APOH mainly regulates hepatic lipid metabolism and is negatively correlated with the prognosis of HCC patients. It is useful for the development of drugs to predict and/or treat the prognosis of fatty liver and end-stage liver disease. The following provides a detailed explanation by examples.

### EXAMPLES

### Example 1. Construction of ApoH Gene Knockout Mice (C57BL/6) and Phenotypic Identification

The construction of ApoH gene knockout mice (C57BL/6) and their phenotypic identification were performed and completed at the Laboratory Animal Center of Xiamen University.

The total procedures were included as below.

S1. Design of the gRNA

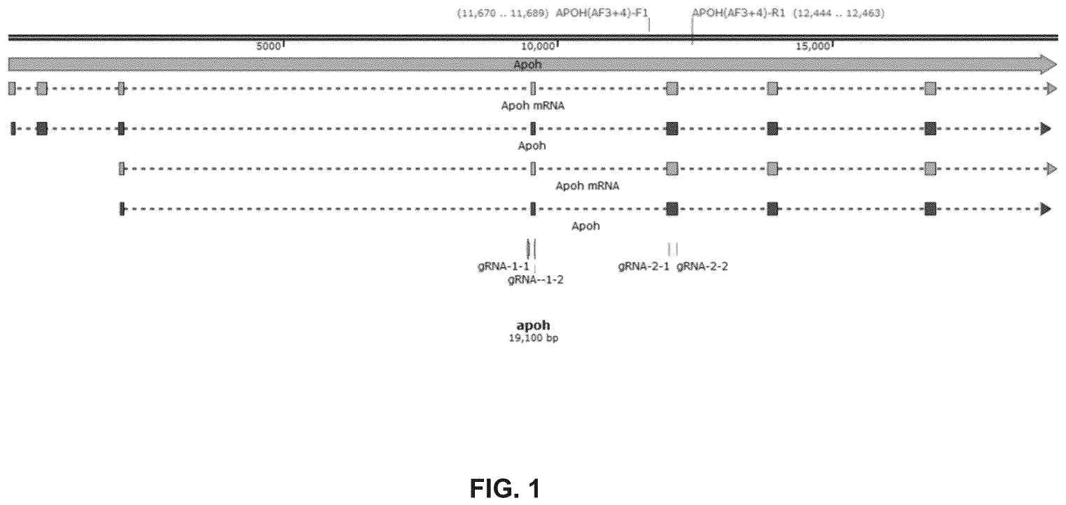

The Mouse ApoH gene had two isoforms, from which the more proximal exons were selected within their common functional region. For example, four gRNAs were designed to target the two exon regions, as shown in FIG. 1.

S2. gRNA Efficiency Assay

Cas9 gRNA plasmids with appropriate resistance were constructed and transfected into 293T cells. Resistant drugs were used to eliminate the non-transfected cells, ensuring that all surviving cells were Cas9 gRNA transfected cells, and the genome was extracted from these cells.

Polymerase chain reaction (PCR) amplification was performed using primers 300 bp before and after the gRNA cleavage site and the target fragments were recovered. The target fragments were then ligated to the vector and spread on the plate, and 16 colonies were selected for sequencing. The proportion of colonies with knockout (KO) characteristic colonies among the selected 16 colonies was counted, and the colonies with a knockout efficiency greater than 50% were selected for the subsequent experiments.

In the example, a pair of gRNAs acting on the fifth exon (exon5) of isoform X1 was finally selected with the following sequences.

(SEQ ID NO. 1)
gRNA-2-1: 5′-TCCAAAGTTTGCACTCCTTA-3′;
(SEQ ID NO. 2)
gRNA-2-2: 5′-GATTGCCAGAATGCCTGGGT-3′.

S3. Order gRNA

2OD of primers were ordered from Thermo Fisher Scientific with the following sequences.

gRNA-2-1:
(SEQ ID NO. 3)
5′-GAAATTAATACGACTCACTATAGGTCCAAAGTTTGCACTCCTTAGTT
TTAGAGCTAGAAATAGC-3′;
gRNA-2-2:
(SEQ ID NO. 4)
5′-GAAATTAATACGACTCACTATAGGGATTGCCAGAATGCCTGGGTGTT
TTAGAGCTAGAAATAGC-3′

The bold and underlined sequences were 5′-3′ gRNA sequences, and the bold and italicized sequences were T7 tag sequences.

S4. Order PCR Primer for Cas9 with the Following Sequences.

(SEQ ID NO. 5)
Cas9-F: 5′-CACCGACTGAGCTCCTTAAG-3′;
(SEQ ID NO. 6)
Cas9-R: 5′-TAGTCAAGCTTCCATGGCTCGA-3′.

### S5. Cytoplasmic Injection

After in vitro transcription of gRNA and Cas9, Cas9 at a ratio of 100 ng/μl and gRNA at a ratio of 50 ng/μl were mixed to obtain a mixture that was microinjected into 0.5-day-old fertilized eggs. A total of 200 fertilized eggs were injected, and then they were transplanted into 10 Institute of Cancer Research (ICR) pseudopregnant female mice. After 19 days, 39 pups were successfully born.

### S6. Mouse Gene Knockout Identification

PCR primers were designed based on the location of the gRNA with sequences as follows.

(SEQ ID NO. 7)
ApoH-F: 5′-TGGCATTGAACTCACACT-3′;
(SEQ ID NO. 8)
ApoH-R: 5′-AACTAAGGCTACACAGAGAA-3′.

Nucleic acid electrophoresis analysis was performed on the resulting PCR products, and 19 samples with significant differences from the WT sequence were selected for sequencing to verify the accuracy of the deleted fragments. These mice were then bred for further study. Currently, mice with a deleted fragment of 140 bp have been used for modeling.

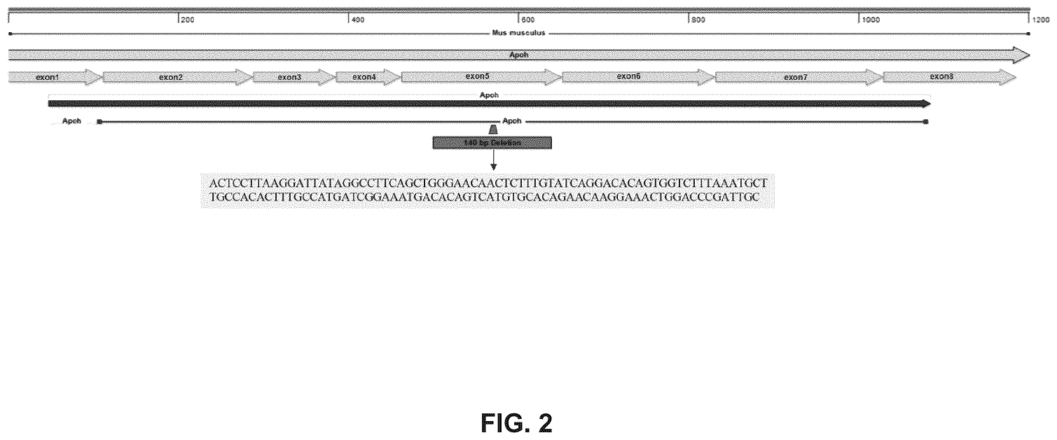

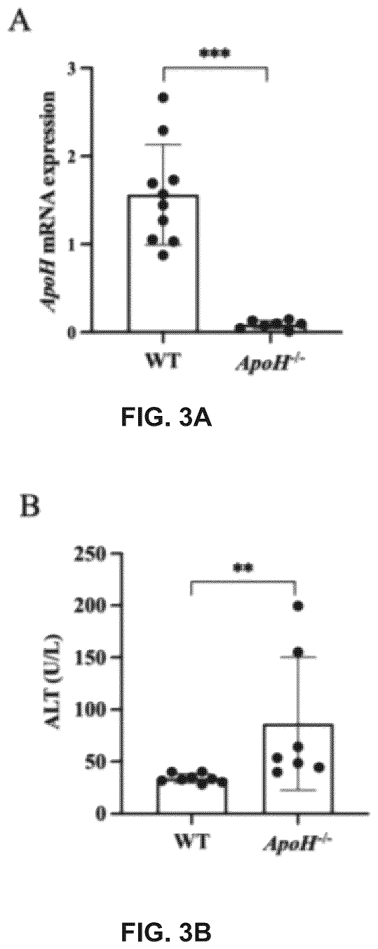

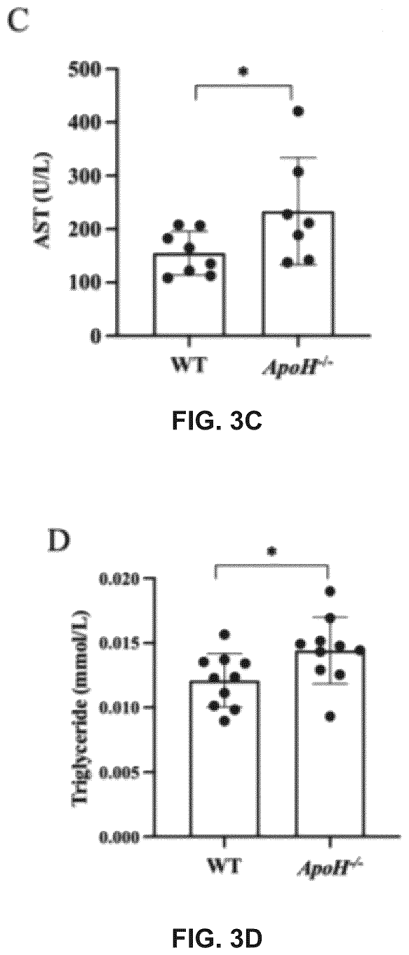

FIG. 2 shows the gene sequence from the liver of an ApoH^{-/-} mouse model according to some embodiments of the present disclosure. FIGS. 3A-3D show the phenotype identification results of the mouse model according to some embodiments of the present disclosure, FIG. 3A shows the RT-qPCR detection result of RNA extracted from the liver of the mice, FIG. 3B shows the ALT level in the peripheral blood of the mouse model, FIG. 3C shows the AST level in the peripheral blood of mouse model, and FIG. 3D shows the TG level in the liver of the mouse model. The above results showed that the ApoH gene was successfully knocked out in mice.

### S7. Phenotypic Identification of Gene Knockout Mice

Transaminase levels: Using WT C57BL/6 mice as controls, the serum transaminase levels in the peripheral blood of 10-week-old ApoH gene knockout mice (C57BL/6 ApoH) were assessed. The results showed that serum ALT and AST levels were significantly increased (FIGS. 3B and 3C).

TG levels: Liver TG levels were further assessed. The results showed that TG levels were significantly increased in the liver of ApoH^{-/-} mice (FIG. 3D).

In summary, ApoH^{-/-} mice exhibited spontaneous steatohepatitis.

### Example 2. Analysis of APOH Gene Levels in the Liver Tissue of Patients with CLDs and HCC and Survival Analysis of HCC Patients

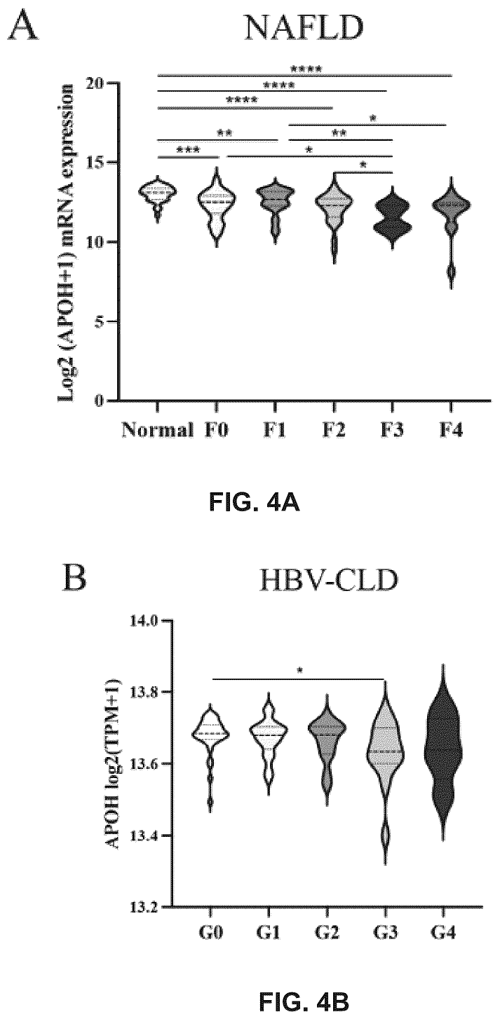

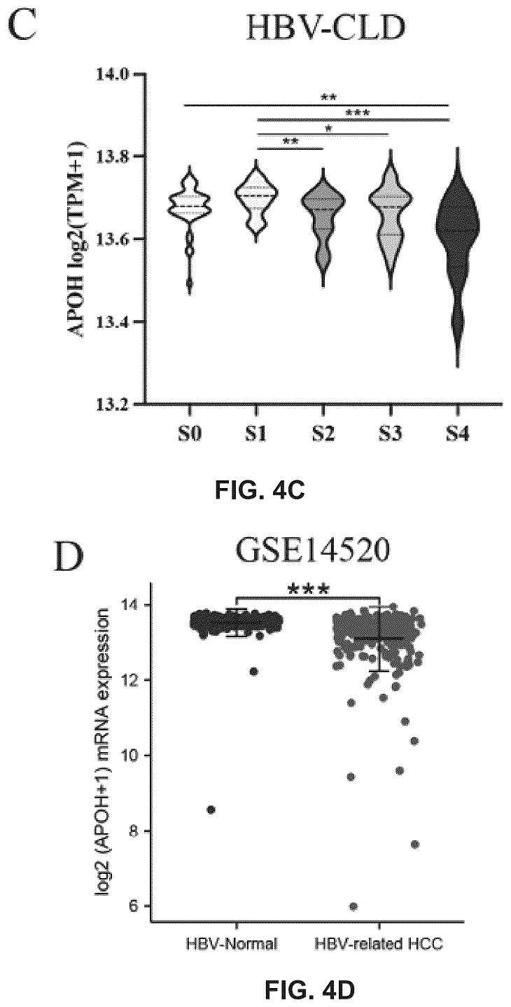

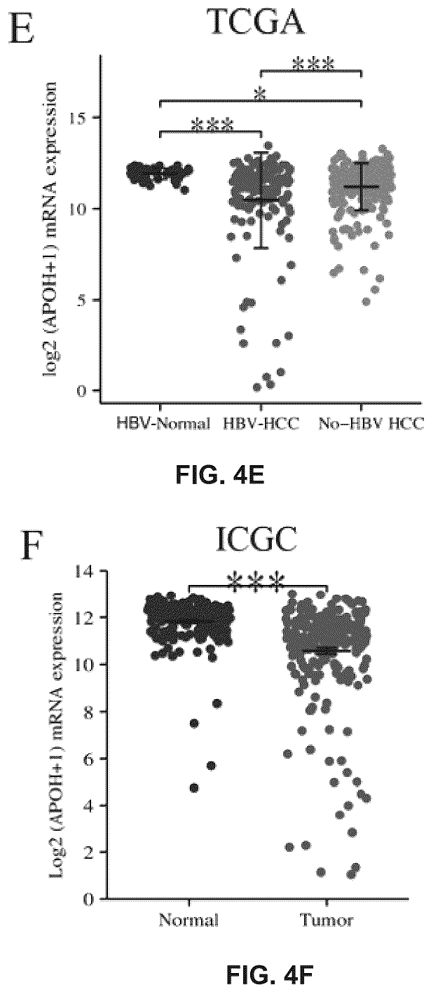

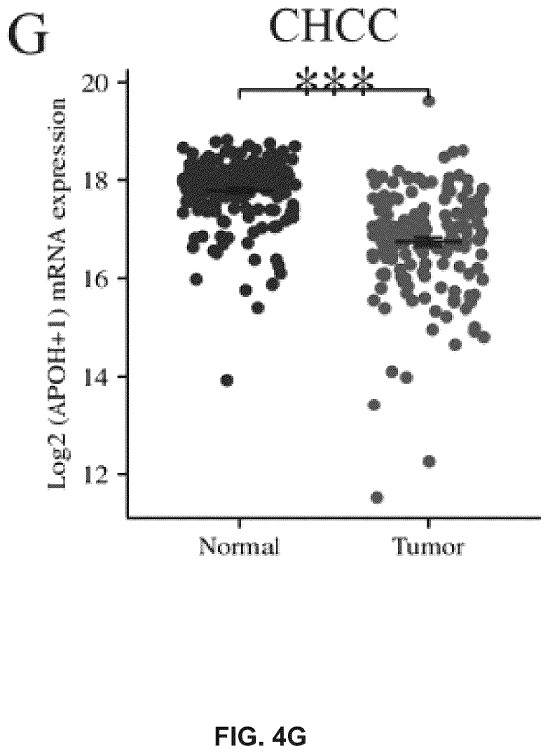

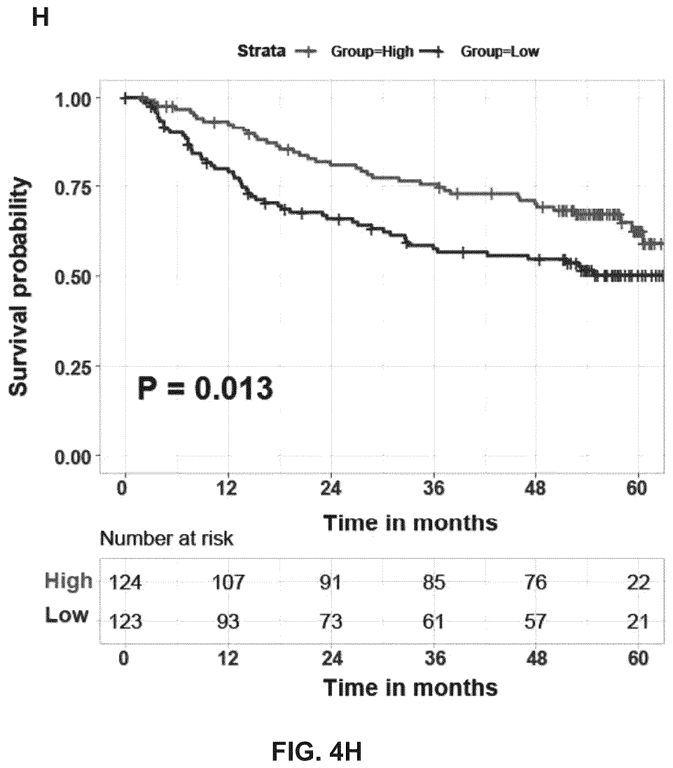

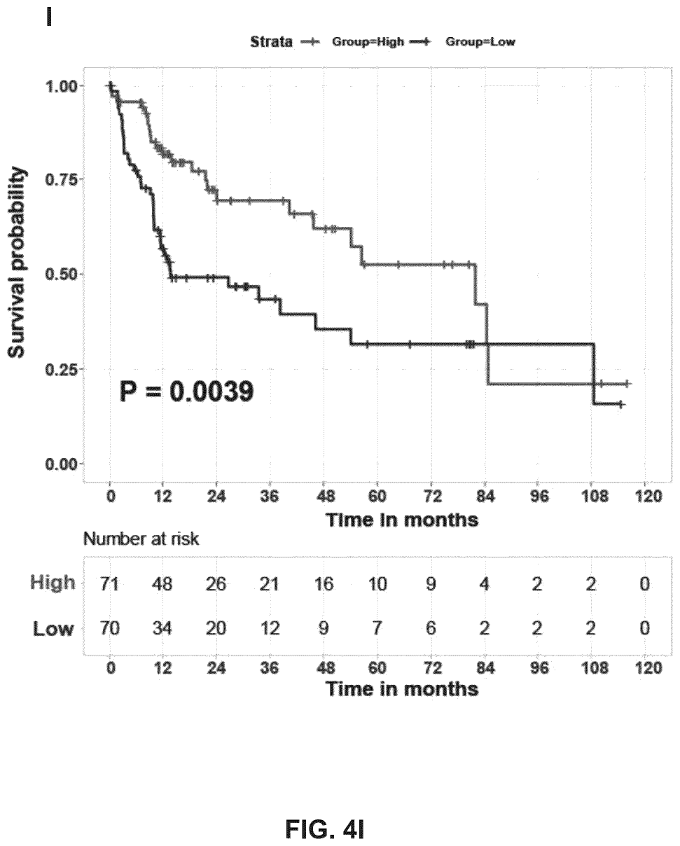

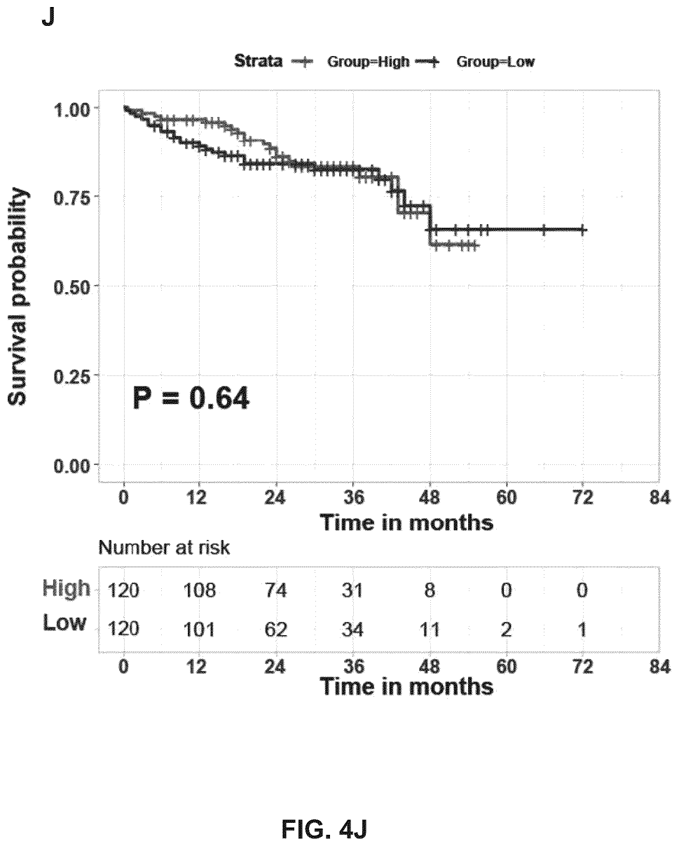

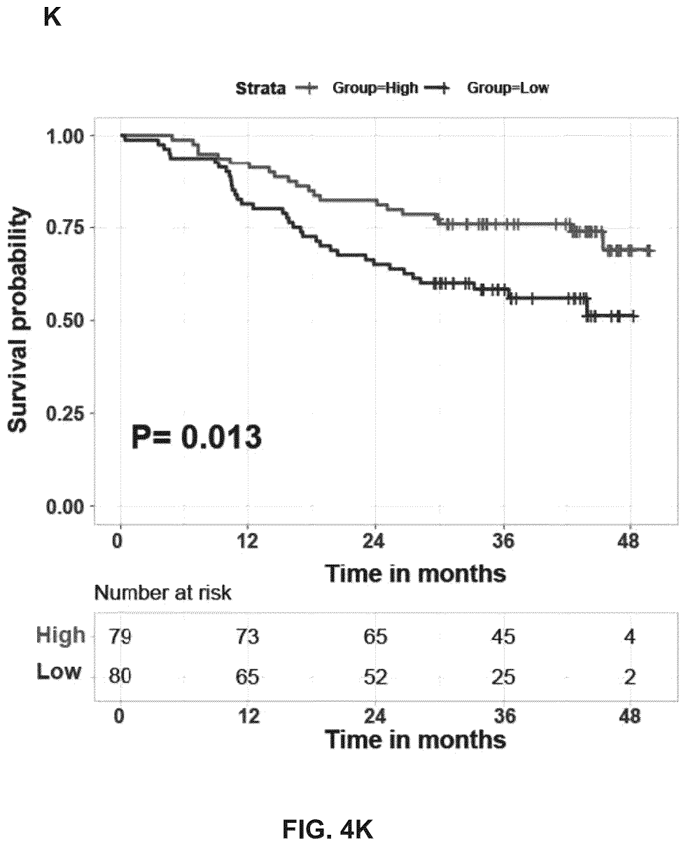

S1. By analyzing the liver transcriptomic datasets from CLDs patients, the results showed that the expression of APOH gene in the liver of patients with NASH was significantly decreased with the progression of liver fibrosis (Table 1), and the expression of the APOH gene in the liver of patients with chronic hepatitis B was significantly decreased with the progression of liver inflammation and liver fibrosis (Table 2). FIGS. 4A-4K show the APOH gene levels in the liver tissues of patients with CLDs and HCC, and the survival analysis of HCC patients according to some embodiments of the present disclosure. FIG. 4A shows the APOH gene levels in the liver tissue from patients with NASH at different stages of liver fibrosis. FIGS. 4B and 4C show the APOH gene levels in the liver tissue from patients with HBV-related chronic liver diseases (HBV-CLDs) at different stages of inflammation and liver fibrosis. FIGS. 4D, 4E, 4F, and 4G show the APOH gene levels in the liver tissue of patients with HBV-related hepatocellular carcinoma (HBV-HCC) in the Gene Expression Omnibus (GEO), the Cancer Genome Atlas (TCGA), International Cancer Genome Consortium (ICGC), and Chinese Hepatocellular Carcinoma Consortium (CHCC) databases, respectively. FIGS. 4H, 4I, 4J, and 4K show the survival differences between the low expression group of APOH gene and the high expression group of APOH gene in the liver tissue of HBV-HCC patients in the GEO, TCGA, ICGC, and CHCC databases, respectively. FIG. 4A showed a significant difference in the APOH gene levels in the liver tissue of NASH patients at different stages of liver fibrosis. FIGS. 4B and 4C indicated that the expression of APOH gene in the liver of patients with HBV-CLDs was significantly decreased with the progression of liver inflammation and liver fibrosis. The difference was statistically significant.

TABLE 1
APOH gene levels in the liver tissue from NASH
patients at different stages of liver fibrosis.

| | Normal | F0 | F1 | F2 | F3 | F4 |
|---|---|---|---|---|---|---|
| Number of | 31 | 35 | 30 | 27 | 8 | 12 |
| cases | | | | | | |
| Gender | 28/3 | 25/10 | 20/10 | 19/8 | 4/4 | 7/5 |
| (Female/Male) | | | | | | |
| APOH gene | 13.020 | 12.297 | 12.514 | 12.110 | 11.532 | 11.743 |
| | (11.684, | (10.465, | (10.580, | (9.524, | (10.520, | (8.117, |
| | 13.685) | 13.921) | 13.362) | 13.477) | 12.530) | 13.011) |

TABLE 2
APOH gene levels in the liver tissue of patients
with chronic hepatitis B in stages G and S.

| | G0 | G1 | G2 | G3 | G4 |
|---|---|---|---|---|---|
| Number of | 37 | 33 | 34 | 15 | 5 |
| cases | | | | | |
| Gender | 10/27 | 8/25 | 15/19 | 3/12 | 0/5 |
| (Female/Male) | | | | | |
| APOH gene | 13.679 | 13.670 | 13.663 | 13.631 | 13.640 |
| | (13.494, | (13.557, | (13.536, | (13.400, | (13.510, |
| | 13.739) | 13.754) | 13.741) | 13.748) | 13.751) |
| | S0 | S1 | S2 | S3 | S4 |
| Number of | 43 | 20 | 33 | 18 | 10 |
| cases | | | | | |
| Gender | 11/32 | 4/16 | 16/17 | 3/15 | 2/8 |
| (Female/Male) | | | | | |
| APOH gene | 13.676 | 13.697 | 13.654 | 13.659 | 13.600 |
| | (13.494, | (13.631, | (13.536, | (13.543, | (13.400, |
| | 13.750) | 13.754) | 13.720) | 13.751) | 13.711) |

S2. Further analysis of the liver transcriptomic datasets from HCC patients revealed that the expression of the APOH gene was significantly decreased in the liver tumor tissue compared to the adjacent normal liver tissues (FIGS. 4D-4G). Furthermore, further survival analysis showed that patients with high APOH expression in the liver tumor tissue had significantly longer survival time compared to those with low APOH expression in the liver tumor tissue, i.e., downregulation of APOH gene was negatively correlated with prognosis, and the differential survival was observed between the low and high APOH expression groups (FIGS. 4H-4K).

In summary, downregulation of APOH-mediated steatohepatitis accelerated the progression of CLDs and closely correlated with the prognosis of HCC patients, which implied the significant clinical relevance in the accurate diagnosis and treatment of MASLD.

### Example 3. Effect of Increasing APOH Expression on Steatohepatitis in Mice

Six-week-old C57BL/6 ApoH^{-/-} (KO) mice and WT mice were used as subjects and fed a 45% high-fat diet for 12 weeks to construct a fatty liver mouse model. After the fatty liver mouse model was constructed, mouse serum and liver samples were collected to measure serum transaminase levels and ApoH gene levels in the liver tissue.

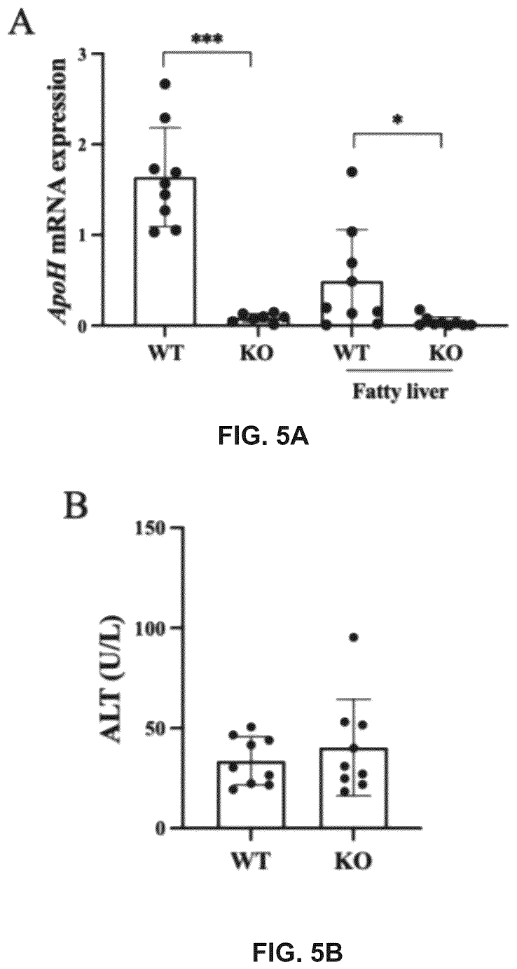

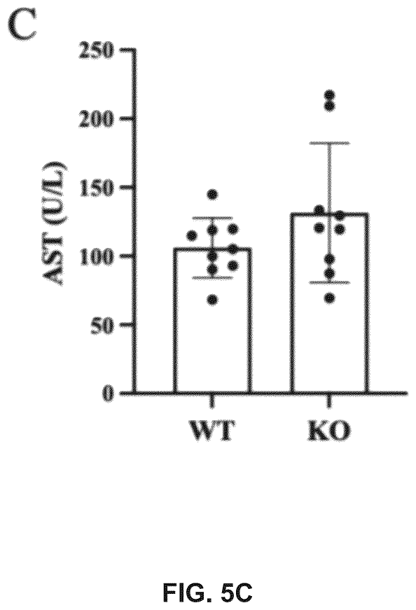

The results were shown in FIGS. 5A-5C. FIGS. 5A-5C show the results of phenotypic identification from the fatty liver mouse model according to some embodiments of the present disclosure. FIG. 5A shows the gene levels in the liver of mice by RT-qPCR assay, FIG. 5B shows the serum ALT level of the mouse model, and FIG. 5C shows the serum AST level the mouse model. In the fatty liver mouse model, the expression of ApoH gene in the liver of WT mice was significantly reduced, and was slightly higher than that in the liver of ApoH^{-/-} mice (FIG. 5A); and no significant difference of serum ALT and AST levels was observed between WT and ApoH^{-/-} mice on the back ground of high-fat diet (FIGS. 5B and 5C).

Therefore, it is further confirmed that ApoH gene deletion drives the hepatic steatosis in mice.

The following methods were performed to upregulate APOH levels in mice and further explore the potential effect on hepatic steatosis and inflammatory injury in the liver.

(I) Exogenous administration of recombinant APOH protein through the tail vein of mice: gradient concentration of recombinant APOH protein were injected into the ApoH gene knockout mice through the tail vein, with the non-injected group as controls. Then, mouse peripheral blood and liver tissues were collected at different time points to measure the serum transaminases and lipids levels, the liver APOH and TG levels, and the degree of liver pathologic steatosis, inflammation, and fibrosis.

(II) Targeting the mouse liver tissue to induce overexpression of the ApoH gene: an adeno-associated virus (AAV)-mediated hepatocyte-specific ApoH gene overexpression model was further constructed using WT C57BL/6 mice. Then, peripheral blood and liver tissues were collected at different time points to measure the serum transaminases and lipid levels, the liver APOH and TG levels, and the degree of liver pathologic steatosis, inflammation, and fibrosis.

(III) Construction of a sterile gut microenvironment mouse model: The ApoH^{-/-} mice were fed an antibiotic complex to create a sterile intestinal environment, and the mice with the native intestinal microenvironment served as controls. The protocol of antibiotic complex was reported by DagHenrik Reikvamd et al. Then, the serum transaminase and lipid levels, hepatic APOH and TG levels, and the degree of liver pathological steatosis, inflammation, and fibrosis were measured.

These experimental results indicated that exogenous supplementation of recombinant APOH protein or maintaining the homeostasis of gut microbiota can effectively alleviate liver inflammatory injury in mice by enhancing the APOH production. These results indicated that serum transaminase level was decreased and the liver pathologic damage, including steatosis, inflammation and fibrosis, was reduced. In summary, the balance of APOH level is important to modulate the progression of CLDs and improve the prognosis of patients with end-stage liver disease, which is valuable for the accurate diagnosis and treatment of MASLD.

In addition, by analyzing the mechanisms and the changes in expression of APOH in MASLD and related diseases, the pattern of change in the expression of APOH during the development of MASLD and related diseases was established. Serum APOH level can be used as a non-invasive biomarker for early diagnosis and severity assessment of HCC. On the other hand, exogenous supplementation of APOH protein and related formulations may alleviate liver injury and slow disease progression by increasing APOH levels.

The technical solutions in the present disclosure explore the role and pattern of change of APOH in the development of MASLD and related diseases, and establish biological diagnostic kits and treatment strategies for different stages of MASLD and related diseases. The increasing APOH levels should be an effective treatment method for patients with MASLD and related diseases. In addition, the technical solutions of the present disclosure also explore the role of APOH in the treatment of MASLD and related diseases.

The basic concepts have been described above, apparently, in detail, as will be described above, and do not constitute limitations of the disclosure. Although there is no clear explanation here, those skilled in the art may make various modifications, improvements, and corrections of the present disclosure. This type of modifications, improvements, and corrections are recommended in the present disclosure, so the modifications, improvements, and the corrections remain in the spirit and scope of the exemplary embodiment of the present disclosure.

At the same time, the present disclosure uses specific words to describe the embodiments of the present disclosure. As “one embodiment”, “an embodiment”, and/or “some embodiments” means a certain feature, structure, or characteristic of at least one embodiment of the present disclosure. Therefore, it is emphasized and should be appreciated that two or more references to “an embodiment” or “one embodiment” or “an alternative embodiment” in various parts of the present disclosure are not necessarily all referring to the same embodiment. Further, certain features, structures, or features of one or more embodiments of the present disclosure may be combined.

In addition, unless clearly stated in the claims, the order of processing elements and sequences, the use of numbers and letters, or the use of other names in the present disclosure are not used to limit the order of the procedures and methods of the present disclosure. Although the above disclosure discusses through various examples what is currently considered to be a variety of useful embodiments of the disclosure, it is to be understood that such detail is solely for that purpose, and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof for the purpose of streamlining the disclosure aiding in the understanding of one or more of the various embodiments. However, this disclosure does not mean that the subject matter of the present disclosure object requires more features than the features mentioned in the claims. Rather, the claimed subject matter may lie in less than all features of a single foregoing disclosed embodiment.

In some embodiments, the numbers expressing quantities of ingredients, properties, and so forth, used to describe and claim certain embodiments of the application are to be understood as being modified in some instances by the terms “about”, “approximate”, or “substantially”. Unless otherwise stated, “about”, “approximate”, or “substantially” may indicate ±20% variation of the value it describes. Accordingly, in some embodiments, the numerical parameters used in the disclosure and claims are approximate values, and the approximation may change according to the characteristics required by the individual embodiments. In some embodiments, the numerical parameter should consider the prescribed effective digits and adopt a general digit retention method. Although in some embodiments, the numerical fields and parameters used to confirm the breadth of its range are approximate values, in specific embodiments, such numerical values are set as accurately as possible within the feasible range.

With respect to each patent, patent application, patent application disclosure, and other material cited in the present disclosure, such as articles, books, manuals, publications, documents, etc., the entire contents thereof are hereby incorporated by reference into the present disclosure. Application history documents that are inconsistent with the contents of the present disclosure or that create conflicts are excluded, as are documents (currently or hereafter appended to the present disclosure) that limit the broadest scope of the claims of the present disclosure. It should be noted that in the event of any inconsistency or conflict between the descriptions, definitions, and/or use of terms in the materials appended to the present disclosure and those described in the present disclosure, the descriptions, definitions, and/or use of terms in the present disclosure shall prevail.

At last, it should be understood that the embodiments described in the present disclosure are merely illustrative of the principles of the embodiments of the present disclosure. Other modifications that may be employed may be within the scope of the present disclosure. Thus, by way of example, but not of limitation, alternative configurations of the embodiments of the present disclosure may be utilized in accordance with the teachings herein. Accordingly, embodiments of the present disclosure are not limited to that precisely as shown and described.

What is claimed is:

1. A use of apolipoprotein H (APOH) in manufacture of a medicament for prevention and/or treatment of fatty liver disease.

2. The use of claim 1, wherein the medicament comprises APOH or APOH gene, and the medicament is used to increase a level of APOH in liver.

3. The use of claim 1, wherein the medicament comprises a medically acceptable excipient, and the medicament is in a form of an injectable, a tablet, a granule, an oral formulation, or a genetically engineered drug.


SEQUENCE LISTING (8 sequences)

SEQ ID NO: 1 - RNA, 20 nt, synthetic construct
  source 1..20: mol_type = other RNA
tccaaagtttgcactcctta

SEQ ID NO: 2 - RNA, 20 nt, synthetic construct
  source 1..20: mol_type = other RNA
gattgccagaatgcctgggt

SEQ ID NO: 3 - RNA, 64 nt, synthetic construct
  source 1..64: mol_type = other RNA
gaaattaatacgactcactataggtccaaagtttgcactccttagttttagagctagaaatagc

SEQ ID NO: 4 - RNA, 64 nt, synthetic construct
  source 1..64: mol_type = other RNA
gaaattaatacgactcactatagggattgccagaatgcctgggtgttttagagctagaaatagc

SEQ ID NO: 5 - RNA, 20 nt, synthetic construct
  source 1..20: mol_type = other RNA
caccgactgagctccttaag

SEQ ID NO: 6 - RNA, 22 nt, synthetic construct
  source 1..22: mol_type = other RNA
tagtcaagcttccatggctcga

SEQ ID NO: 7 - RNA, 18 nt, synthetic construct
  source 1..18: mol_type = other RNA
tggcattgaactcacact

SEQ ID NO: 8 - RNA, 20 nt, synthetic construct
  source 1..20: mol_type = other RNA
aactaaggctacacagagaa